(19) United States
(12) Patent Application Publication
(10) Pub. No.: US 2018/0298104 A1
(43) Pub. Date: Oct. 18, 2018

(54) METHODS AND PHARMACEUTICAL COMPOSITIONS FOR THE TREATMENT OF TH17 MEDIATED DISEASES

(71) Applicants: INSERM (INSTITUT NATIONAL DE LA SANTE ET DE LA RECHERCHE MEDICALE), Paris (FR); UNIVERSITY OF NOTTINGHAM, Nottingham, Nottinghamshire (GB); UNIVERSITE DE BORDEAUX, Bordeaux (FR); CHU HOPITAUX DE BORDEAUX, Bordeaux (FR); UNIVERSITE DE RENNES, Rennes (FR); CENTRE NATIONAL DE LA RECHERCHE SCIENTIFIQUE(CNRS), Paris (FR)

(72) Inventors: Patrick LEGEMBRE, Rennes (FR); Patrick BLANCO, Bordeaux (FR); Robin FLYNN, Sutton Bonington Leicestershire (GB)

(21) Appl. No.: 15/568,205

(22) PCT Filed: Apr. 21, 2016
(86) PCT No.: PCT/EP2016/058838

§ 371 Date: Oct. 20, 2017

(30) Foreign Application Priority Data
Apr. 22, 2015 (EP) 15305606.4

(51) Int. Cl. C07K 16/28; C12N 15/115; C12N 15/113

(52) U.S. Cl. CPC C07K 16/2875; C12N 15/115; C12N 2310/14; C12N 2310/16; C12N 15/1138

(57) ABSTRACT

The present invention relates to methods and pharmaceutical compositions for the treatment of Th17-mediated diseases. In particular, the present invention relates to a method of treating a Th17-mediated disease in a subject in need thereof comprising administering to the subject a therapeutically effective amount of a CD95 antagonist.

### FIELD OF THE INVENTION

The present invention relates to methods and pharmaceutical compositions for the treatment of Th17-mediated diseases.

### BACKGROUND OF THE INVENTION

CD95L (FasL) belongs to the Tumour Necrosis Factor (TNF) family and is the ligand of the death receptor CD95 (also known as Fas). While CD95 is ubiquitously expressed on healthy cells, CD95L exhibits a restricted expression pattern, mainly detected on the surface of lymphocytes, where it plays a pivotal role in the elimination of infected and transformed cells. CD95L can also be expressed on epithelial cells, macrophages and dendritic cells under inflammatory conditions (Fouque et al., 2014) and on the tumor endothelium in human and mouse (Malleter et al., 2013; Motz et al., 2014). CD95L is a 37 kDa type II transmembrane glycoprotein that acts locally through cell-to-cell contact (Suda et al., 1993). The extracellular domain of human CD95L is composed of a juxtamembrane stalk region (103-136 aa) and a TNF homology domain (137-281 aa) (Orlinick et al., 1997). This stalk region can be cleaved by metalloproteases such as MMP3 (Matsuno et al., 2001), MMPI (Vargo-Gogola et al., 2002), MMP9 (Kiaei et al., 2007) or A Disintegrin And Metalloproteinase 10 (ADAM-10) (Kirkin et al., 2007; Schulte et al., 2007) and thus releasing CD95L into the bloodstream. This soluble ligand contributes to aggravating inflammation in chronic inflammatory disorders such as systemic lupus erythematosus (SLE) (O'Reilly et al., 2009; Tauzin et al., 2011) by inducing non-apoptotic signaling pathways such as NF-κB (O'Reilly et al., 2006) and PI3K (Tauzin et al., 2011). While in presence of membrane-bound CD95L, the intracellular region of CD95 designated the death domain (DD) orchestrates the formation of the death inducing signaling complex (DISC) by recruiting the adaptor molecule FADD, which in turn causes aggregation of caspase-8 inducing apoptosis (Kischkel et al., 1995), the non-apoptotic signaling pathway triggered by cleaved CD95L (cl-CD95L) implements the formation of a different complex termed motility-inducing signaling complex (MISC) through tyrosine kinase phosphorylation and a Ca²⁺ influx response (Kleber et al., 2008; Malleter et al., 2013; Tauzin et al., 2011). The complete composition of the MISC and more importantly the molecular mechanisms by which CD95 switches from inducing non-apoptotic to apoptotic signaling pathways remain to be elucidated (Fouque et al., 2014).

SLE is a chronic autoimmune disorder affecting almost all organs and tissues whose etiology and pathogenesis remain largely unknown. A body of evidence in human and mouse studies supports a role for the cytokine interleukin-17 (IL-17) and IL-17 producing Th17 cells in the pathogenesis of SLE disease (for review see (Shin et al., 2011)). A high percentage of CD4⁺ T-cells and an increased number of blood CD3⁺ CD4⁻ CD8⁻ T cells in SLE patients produce IL-17, and these cell types home to the kidney in patients with lupus nephritis (Crispin et al., 2008; Wang et al., 2010). SLE patients also exhibit a marked infiltration of Th17 T cells secreting cytokines in their skin (Yang et al., 2009). Mechanistically, a study of a murine model of acute glomerulonephritis, demonstrated that cxcr3^{-/-} animals are partly protected from immunopathology by a reduction in renal Th17 cell accumulation (Steinmetz et al., 2009). Accordingly, dysfunction of Th17 lymphocytes trafficking to organs may participate in the pathogenesis of SLE and their modulation holds promise as an attractive therapeutic approach to reduce the inflammatory process. From a mechanistic perspective, the mechanisms responsible for the differentiation and stabilization of these T-cells are clearly understood; conversely there is little insight to explain how Th17 lymphocytes accumulate in the damaged organs of SLE patients.

### SUMMARY OF THE INVENTION

The present invention relates to methods and pharmaceutical compositions for the treatment of Th17-mediated diseases. In particular, the present invention is defined by the claims.

### DETAILED DESCRIPTION OF THE INVENTION

High serum concentrations of soluble CD95L are correlated with the severity of the pathology in systemic lupus erythematosus (SLE) patients. This soluble CD95L is able to enhance extravasation of activated T cells, a cellular phenomenon contributing to the accumulation of lymphocytes in inflamed tissues. Now the inventors demonstrate that CD95L is over-expressed in endothelial cells in inflamed skin of lupus patients. In addition, after cleavage by metalloprotease, cleaved-CD95L (cl-CD95L) promotes endothelial transmigration of human and murine T-helper (Th) 17 lymphocytes at the expense of T-regulatory (Treg) lymphocytes. Activated T-cell transmigration is achieved through a CD95-mediated Ca²⁺ signal. By using neutralizing antibodies, or decoy receptor polypeptides, the invention prevent in vitro endothelial transmigration of Th17 lymphocytes. This study thus provides novel insights into the cellular and molecular mechanisms by which cl-CD95L contributes to SLE pathogenesis. Moreover, neutralizing the CD95/CD95L signaling pathway turns out to be a very attractive therapeutic approach to treat SLE patients but in general Th17 mediated diseases by preventing damages caused by the accumulation of activated Th17 cells in organs.

Accordingly, one aspect of the present invention relates to a method of treating a Th17 mediated disease in a subject in need thereof comprising administering to the subject a therapeutically effective amount of a CD95 antagonist.

As used herein, the expression “Th17-mediated disease” is used herein in the broadest sense and includes all diseases and pathological conditions the pathogenesis of which involves abnormalities of Th17 cells, in particulate accumulation of Th17 cells in organs. As used herein, the term “Th17 cells” has its general meaning in the art and refers to a subset of T helper cells producing interleukin 17 (IL-17). “A brief history of T(H)17, the first major revision in the T(H)1/T(H)2 hypothesis of T cell-mediated tissue damage”. Nat. Med. 13 (2): 139-145.). The term “IL-17” has its general meaning in the art and refers to the interleukin-17A protein. Typically, Th17 cells are characterized by classical expression of Th cell markers at their cell surface such as CD4, and by the expression of IL17. Typically, as referenced herein, a Th17 cell is a IL-17+ cell.

Examples of Th17 mediated diseases include but are not limited to autoimmune diseases, inflammatory diseases, osteoclasia, and transplantation rejection of cells, tissue and organs. In particular, the above-mentioned Th17-mediated diseases may be one or more selected from the group consisting of Behçet's disease, polymyositis/dermatomyositis, autoimmune cytopenias, autoimmune myocarditis, primary liver cirrhosis, Goodpasture's syndrome, autoimmune meningitis, Sjögren's syndrome, systemic lupus erythematosus, Addison's disease, alopecia greata, ankylosing spondylitis, autoimmune hepatitis, autoimmune mumps, Crohn's disease, insulin-dependent diabetes mellitus, dystrophic epidermolysis bullosa, epididymitis, glomerulonephritis, Graves' disease, Guillain-Barré syndrome, Hashimoto's disease, hemolytic anemia, multiple sclerosis, myasthenia gravis, pemphigus vulgaris, psoriasis, rheumatic fever, rheumatoid arthritis, sarcoidosis, scleroma, spondyloarthropathy, thyroiditis, vasculitis, vitiligo, myxedema, pernicious anemia and ulcerative colitis.

In some embodiments, the CD95 antagonist of the present invention is particularly suitable for preventing endothelial transmigration of activated Th17 cells, preventing accumulation of activated Th17 cells in organs and preventing damages caused by accumulation of activated Th17 cells in organs.

As used herein, the term “CD95” has its general meaning in the art and refers to CD95, the receptor present on the surface of mammalian cells, which has been originally shown to have the capacity to induce apoptosis upon binding of the trimeric form of its cognate ligand, CD95L (Krammer, P. H. (2000). CD95's deadly mission in the immune system. Nature 407, 789-795). CD95 is also known as FasR or Apo-1. An exemplary amino acid sequence of CD95 is shown by UniProtKB/Swiss-Prot accession number: P25445.

As used herein the term “CD95L” has its general meaning in the art and refers to the cognate ligand of CD95 that is a transmembrane protein. As used herein the term “soluble CD95L” has its general meaning in the art and refers to the soluble ligand produced by the cleavage of the transmembrane CD95L (also known as FasL) (Matsuno et al., 2001; Vargo-Gogola et al., 2002; Kiaei et al., 2007; Kirkin et al., 2007; or Schulte et al., 2007). The term “serum CD95L”, “soluble CD95L”, “metalloprotease-cleaved CD95L” and “cl-CD95L” have the same meaning along the specification. An exemplary amino acid sequence of CD95L is shown by UniProtKB/Swiss-Prot accession number: P48023)

The term “CD95 antagonist” means any molecule that attenuates signal transduction mediated by the binding of CD95 to the soluble CD95L. In particular the CD95 antagonist is a molecule that inhibits, reduces or abolishes the transmigration of Th17 cells. In particular the CD95 antagonist is a molecule that inhibits, reduces or abolishes CD95-mediated Ca2+ signal. Such inhibition may result where: (i) the CD95 antagonist of the invention binds to a CD95 without triggering signal transduction, to reduce or block signal transduction mediated by soluble CD95L; (ii) the CD95 antagonist binds to the soluble CD95L, preventing its binding to CD95; (iii) the CD95 antagonist binds to, or otherwise inhibits the activity of, a molecule that is part of a regulatory chain that, when not inhibited, has the result of stimulating or otherwise facilitating CD95 signal transduction mediated by soluble CD95L; or (iv) the CD95 antagonist inhibits CD95 expression or CD95L expression, especially by reducing or abolishing expression of one or more genes encoding CD95 or CD95L. Typically, the CD95 antagonist includes but is not limited to an antibody, a small organic molecule, a polypeptide and an aptamer.

In some embodiments, the agent is an antibody. The invention embraces antibodies or fragments of antibodies. Typically the antibodies of the invention have the ability to block the interaction between soluble CD95L and CD95 or have the ability to block the induction of the signaling pathway mediated by soluble CD95L. The antibodies may have specificity to soluble CD95L or CD95.

In some embodiments, the antibodies or fragment of antibodies are directed to all or a portion of the extracellular domain of CD95. In some embodiments, the antibodies or fragment of antibodies are directed to an extracellular domain of CD95. More particularly this invention provides an antibody or portion thereof capable of inhibiting binding of CD95 to soluble CD95L, which antibody binds to an epitope located within a region of CD95, which region of CD95 binds to soluble CD95L. Even more particularly, the invention provides an antibody or portion thereof capable of binding to an epitope located within a region of CD95, which region of CD95 is involved the oligomerisation of the receptor. Typically, the antibody binds to the cysteine-rich domain 1 of CD95 which is called the pre-ligand assembly domain (PLAD) (Edmond V, Ghali B, Penna A, Taupin J L, Daburon S, Moreau J F, Legembre P. Precise mapping of the CD95 pre-ligand assembly domain. PLoS One. 2012; 7(9):e46236. doi: 10.1371/journal.pone.0046236. Epub 2012 Sep. 25.). In particular the antibody of the invention binds to the regions delimitated between the amino acid at position 43 and the amino acid at position 66.

In some embodiments of the antibodies or portions thereof described herein, the antibody is a monoclonal antibody. In some embodiments of the antibodies or portions thereof described herein, the antibody is a polyclonal antibody. In some embodiments of the antibodies or portions thereof described herein, the antibody is a humanized antibody. In some embodiments of the antibodies or portions thereof described herein, the antibody is a chimeric antibody. In some embodiments of the antibodies or portions thereof described herein, the portion of the antibody comprises a light chain of the antibody. In some embodiments of the antibodies or portions thereof described herein, the portion of the antibody comprises a heavy chain of the antibody. In some embodiments of the antibodies or portions thereof described herein, the portion of the antibody comprises a Fab portion of the antibody. In some embodiments of the antibodies or portions thereof described herein, the portion of the antibody comprises a F(ab′)2 portion of the antibody. In some embodiments of the antibodies or portions thereof described herein, the portion of the antibody comprises a Fc portion of the antibody. In some embodiments of the antibodies or portions thereof described herein, the portion of the antibody comprises a Fv portion of the antibody. In some embodiments of the antibodies or portions thereof described herein, the portion of the antibody comprises a variable domain of the antibody. In some embodiments of the antibodies or portions thereof described herein, the portion of the antibody comprises one or more CDR domains of the antibody.

As used herein, “antibody” includes both naturally occurring and non-naturally occurring antibodies. Specifically, “antibody” includes polyclonal and monoclonal antibodies, and monovalent and divalent fragments thereof. Furthermore, “antibody” includes chimeric antibodies, wholly synthetic antibodies, single chain antibodies, and fragments thereof. The antibody may be a human or nonhuman antibody. A nonhuman antibody may be humanized by recombinant methods to reduce its immunogenicity in man.

Antibodies are prepared according to conventional methodology. Monoclonal antibodies may be generated using the method of Kohler and Milstein (Nature, 256:495, 1975). To prepare monoclonal antibodies useful in the invention, a mouse or other appropriate host animal is immunized at suitable intervals (e.g., twice-weekly, weekly, twice-monthly or monthly) with antigenic forms of soluble CD95L, or CD95. The animal may be administered a final “boost” of antigen within one week of sacrifice. It is often desirable to use an immunologic adjuvant during immunization. Suitable immunologic adjuvants include Freund's complete adjuvant, Freund's incomplete adjuvant, alum, Ribi adjuvant, Hunter's Titermax, saponin adjuvants such as QS21 or Quil A, or CpG-containing immunostimulatory oligonucleotides. Other suitable adjuvants are well-known in the field. The animals may be immunized by subcutaneous, intraperitoneal, intramuscular, intravenous, intranasal or other routes. A given animal may be immunized with multiple forms of the antigen by multiple routes. Briefly, the recombinant soluble CD95L may be provided by expression with recombinant cell lines. CD95 may be provided in the form of human cells expressing CD95 at their surface. Recombinant forms of CD95 or soluble CD95L may be provided using any previously described method. Following the immunization regimen, lymphocytes are isolated from the spleen, lymph node or other organ of the animal and fused with a suitable myeloma cell line using an agent such as polyethylene glycol to form a hydridoma. Following fusion, cells are placed in media permissive for growth of hybridomas but not the fusion partners using standard methods, as described (Coding, Monoclonal Antibodies: Principles and Practice: Production and Application of Monoclonal Antibodies in Cell Biology, Biochemistry and Immunology, 3rd edition, Academic Press, New York, 1996). Following culture of the hybridomas, cell supernatants are analyzed for the presence of antibodies of the desired specificity, i.e., that selectively bind the antigen. Suitable analytical techniques include ELISA, flow cytometry, immunoprecipitation, and western blotting. Other screening techniques are well-known in the field. Preferred techniques are those that confirm binding of antibodies to conformationally intact, natively folded antigen, such as non-denaturing ELISA, flow cytometry, and immunoprecipitation.

Significantly, as is well-known in the art, only a small portion of an antibody molecule, the paratope, is involved in the binding of the antibody to its epitope (see, in general, Clark, W. R. (1986) The Experimental Foundations of Modern Immunology Wiley & Sons, Inc., New York; Roitt, I. (1991) Essential Immunology, 7th Ed., Blackwell Scientific Publications, Oxford). The Fc′ and Fc regions, for example, are effectors of the complement cascade but are not involved in antigen binding. An antibody from which the pFc′ region has been enzymatically cleaved, or which has been produced without the pFc′ region, designated an F(ab′)2 fragment, retains both of the antigen binding sites of an intact antibody. Similarly, an antibody from which the Fc region has been enzymatically cleaved, or which has been produced without the Fc region, designated a Fab fragment, retains one of the antigen binding sites of an intact antibody molecule. Proceeding further, Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Within the antigen-binding portion of an antibody, as is well-known in the art, there are complementarity determining regions (CDRs), which directly interact with the epitope of the antigen, and framework regions (FRs), which maintain the tertiary structure of the paratope (see, in general, Clark, 1986; Roitt, 1991). In both the heavy chain Fd fragment and the light chain of IgG immunoglobulins, there are four framework regions (FR1 through FR4) separated respectively by three complementarity determining regions (CDR1 through CDRS). The CDRs, and in particular the CDRS regions, and more particularly the heavy chain CDRS, are largely responsible for antibody specificity.

It is now well-established in the art that the non CDR regions of a mammalian antibody may be replaced with similar regions of conspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody.

This invention provides in certain embodiments compositions and methods that include humanized forms of antibodies. As used herein, “humanized” describes antibodies wherein some, most or all of the amino acids outside the CDR regions are replaced with corresponding amino acids derived from human immunoglobulin molecules. Methods of humanization include, but are not limited to, those described in U.S. Pat. Nos. 4,816,567, 5,225,539, 5,585,089, 5,693,761, 5,693,762 and 5,859,205, which are hereby incorporated by reference. The above U.S. Pat. Nos. 5,585,089 and 5,693,761, and WO 90/07861 also propose four possible criteria which may used in designing the humanized antibodies. The first proposal was that for an acceptor, use a framework from a particular human immunoglobulin that is unusually homologous to the donor immunoglobulin to be humanized, or use a consensus framework from many human antibodies. The second proposal was that if an amino acid in the framework of the human immunoglobulin is unusual and the donor amino acid at that position is typical for human sequences, then the donor amino acid rather than the acceptor may be selected. The third proposal was that in the positions immediately adjacent to the 3 CDRs in the humanized immunoglobulin chain, the donor amino acid rather than the acceptor amino acid may be selected. The fourth proposal was to use the donor amino acid reside at the framework positions at which the amino acid is predicted to have a side chain atom within 3A of the CDRs in a three dimensional model of the antibody and is predicted to be capable of interacting with the CDRs. The above methods are merely illustrative of some of the methods that one skilled in the art could employ to make humanized antibodies. One of ordinary skill in the art will be familiar with other methods for antibody humanization.

In some embodiments of the humanized forms of the antibodies, some, most or all of the amino acids outside the CDR regions have been replaced with amino acids from human immunoglobulin molecules but where some, most or all amino acids within one or more CDR regions are unchanged. Small additions, deletions, insertions, substitutions or modifications of amino acids are permissible as long as they would not abrogate the ability of the antibody to bind a given antigen. Suitable human immunoglobulin molecules would include IgG1, IgG2, IgG3, IgG4, IgA and IgM molecules. A “humanized” antibody retains a similar antigenic specificity as the original antibody. However, using certain methods of humanization, the affinity and/or specificity of binding of the antibody may be increased using methods of “directed evolution”, as described by Wu et al., J. Mol. Biol. 294:151, 1999, the contents of which are incorporated herein by reference.

Fully human monoclonal antibodies also can be prepared by immunizing mice transgenic for large portions of human immunoglobulin heavy and light chain loci. See, e.g., U.S. Pat. Nos. 5,591,669, 5,598,369, 5,545,806, 5,545,807, 6,150,584, and references cited therein, the contents of which are incorporated herein by reference. These animals have been genetically modified such that there is a functional deletion in the production of endogenous (e.g., murine) antibodies. The animals are further modified to contain all or a portion of the human germ-line immunoglobulin gene locus such that immunization of these animals will result in the production of fully human antibodies to the antigen of interest. Following immunization of these mice (e.g., XenoMouse (Abgenix), HuMAb mice (Medarex/GenPharm)), monoclonal antibodies can be prepared according to standard hybridoma technology. These monoclonal antibodies will have human immunoglobulin amino acid sequences and therefore will not provoke human anti-mouse antibody (KAMA) responses when administered to humans.

In vitro methods also exist for producing human antibodies. These include phage display technology (U.S. Pat. Nos. 5,565,332 and 5,573,905) and in vitro stimulation of human B cells (U.S. Pat. Nos. 5,229,275 and 5,567,610). The contents of these patents are incorporated herein by reference.

Thus, as will be apparent to one of ordinary skill in the art, the present invention also provides for F(ab′) 2 Fab, Fv and Fd fragments; chimeric antibodies in which the Fc and/or FR and/or CDR1 and/or CDR2 and/or light chain CDR3 regions have been replaced by homologous human or non-human sequences; chimeric F(ab′)2 fragment antibodies in which the FR and/or CDR1 and/or CDR2 and/or light chain CDR3 regions have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or non-human sequences. The present invention also includes so-called single chain antibodies. The various antibody molecules and fragments may derive from any of the commonly known immunoglobulin classes, including but not limited to IgA, secretory IgA, IgE, IgG and IgM. IgG subclasses are also well known to those in the art and include but are not limited to human IgG1, IgG2, IgG3 and IgG4.

In some embodiments, the antibody according to the invention is a single domain antibody. The term “single domain antibody” (sdAb) or “VHH” refers to the single heavy chain variable domain of antibodies of the type that can be found in Camelid mammals which are naturally devoid of light chains. Such VHH are also called “Nanobody®”. According to the invention, sdAb can particularly be llama sdAb.

In some embodiments, the CD95 antagonist of the present invention is a polypeptide. In some embodiments the polypeptide is a functional equivalent of CD95. As used herein, a “functional equivalent of CD95 is a compound which is capable of binding to soluble CD95L, thereby preventing its interaction with CD95. The term “functional equivalent” includes fragments, mutants, and muteins of CD95. The term “functionally equivalent” thus includes any equivalent of CD95 obtained by altering the amino acid sequence, for example by one or more amino acid deletions, substitutions or additions such that the protein analogue retains the ability to bind to soluble CD95L. Amino acid substitutions may be made, for example, by point mutation of the DNA encoding the amino acid sequence. Functional equivalents include molecules that bind soluble CD95L and comprise all or a portion of the extracellular domains of CD95. The functional equivalents include soluble forms of the CD95. A suitable soluble form of these proteins, or functional equivalents thereof, might comprise, for example, a truncated form of the protein from which the transmembrane domain has been removed by chemical, proteolytic or recombinant methods. Preferably, the functional equivalent is at least 80% homologous to the corresponding protein. In a preferred embodiment, the functional equivalent is at least 90% homologous as assessed by any conventional analysis algorithm such as for example, the Pileup sequence analysis software (Program Manual for the Wisconsin Package, 1996).

As used herein, the term “a functionally equivalent fragment” as used herein also may mean any fragment or assembly of fragments of CD95 that binds to soluble CD95L. Accordingly the present invention provides a polypeptide capable of inhibiting binding of CD95 to soluble CD95L, which polypeptide comprises consecutive amino acids having a sequence which corresponds to the sequence of at least a portion of an extracellular domain of CD95, which portion binds to soluble CD95L. In some embodiments, the polypeptide corresponds to an extracellular domain of CD95.

Functionally equivalent fragments may belong to the same protein family as the human CD95 identified herein. By “protein family” is meant a group of proteins that share a common function and exhibit common sequence homology. Homologous proteins may be derived from non-human species. Preferably, the homology between functionally equivalent protein sequences is at least 25% across the whole of amino acid sequence of the complete protein. More preferably, the homology is at least 50%, even more preferably 75% across the whole of amino acid sequence of the protein or protein fragment. More preferably, homology is greater than 80% across the whole of the sequence. More preferably, homology is greater than 90% across the whole of the sequence. More preferably, homology is greater than 95% across the whole of the sequence.

The polypeptides of the invention may be produced by any suitable means, as will be apparent to those of skill in the art. In order to produce sufficient amounts of CD95 or functional equivalents thereof for use in accordance with the present invention, expression may conveniently be achieved by culturing under appropriate conditions recombinant host cells containing the polypeptide of the invention. Preferably, the polypeptide is produced by recombinant means, by expression from an encoding nucleic acid molecule. Systems for cloning and expression of a polypeptide in a variety of different host cells are well known. When expressed in recombinant form, the polypeptide is preferably generated by expression from an encoding nucleic acid in a host cell. Any host cell may be used, depending upon the individual requirements of a particular system. Suitable host cells include bacteria mammalian cells, plant cells, yeast and baculovirus systems. Mammalian cell lines available in the art for expression of a heterologous polypeptide include Chinese hamster ovary cells. HeLa cells, baby hamster kidney cells and many others. Bacteria are also preferred hosts for the production of recombinant protein, due to the ease with which bacteria may be manipulated and grown. A common, preferred bacterial host is E coli.

In specific embodiments, it is contemplated that polypeptides used in the therapeutic methods of the present invention may be modified in order to improve their therapeutic efficacy. Such modification of therapeutic compounds may be used to decrease toxicity, increase circulatory time, or modify biodistribution. For example, the toxicity of potentially important therapeutic compounds can be decreased significantly by combination with a variety of drug carrier vehicles that modify biodistribution. In example adding dipeptides can improve the penetration of a circulating agent in the eye through the blood retinal barrier by using endogenous transporters. A strategy for improving drug viability is the utilization of water-soluble polymers. Various water-soluble polymers have been shown to modify biodistribution, improve the mode of cellular uptake, change the permeability through physiological barriers; and modify the rate of clearance from the body. To achieve either a targeting or sustained-release effect, water-soluble polymers have been synthesized that contain drug moieties as terminal groups, as part of the backbone, or as pendent groups on the polymer chain. Polyethylene glycol (PEG) has been widely used as a drug carrier, given its high degree of biocompatibility and ease of modification. Attachment to various drugs, proteins, and liposomes has been shown to improve residence time and decrease toxicity. PEG can be coupled to active agents through the hydroxyl groups at the ends of the chain and via other chemical methods; however, PEG itself is limited to at most two active agents per molecule. In a different approach, copolymers of PEG and amino acids were explored as novel biomaterials which would retain the biocompatibility properties of PEG, but which would have the added advantage of numerous attachment points per molecule (providing greater drug loading), and which could be synthetically designed to suit a variety of applications. Those of skill in the art are aware of PEGylation techniques for the effective modification of drugs. In addition, to the polymer backbone being important in maintaining circulatory half-life, and biodistribution, linkers may be used to maintain the therapeutic agent in a pro-drug form until released from the backbone polymer by a specific trigger, typically enzyme activity in the targeted tissue. For example, this type of tissue activated drug delivery is particularly useful where delivery to a specific site of biodistribution is required and the therapeutic agent is released at or near the site of pathology Linking group libraries for use in activated drug delivery are known to those of skill in the art and may be based on enzyme kinetics, prevalence of active enzyme, and cleavage specificity of the selected disease-specific enzymes. Such linkers may be used in modifying the protein or fragment of the protein described herein for therapeutic delivery.

In some embodiments, the polypeptides of the invention may be fused to a heterologous polypeptide (i.e. polypeptide derived from an unrelated protein, for example, from an immunoglobulin protein).

As used herein, the terms “fused” and “fusion” are used interchangeably. These terms refer to the joining together of two more elements or components, by whatever means including chemical conjugation or recombinant means. An “in-frame fusion” refers to the joining of two or more polynucleotide open reading frames (ORFs) to form a continuous longer ORF, in a manner that maintains the correct translational reading frame of the original ORFs. Thus, a recombinant fusion protein is a single protein containing two or more segments that correspond to polypeptides encoded by the original ORFs (which segments are not normally so joined in nature. Although the reading frame is thus made continuous throughout the fused segments, the segments may be physically or spatially separated by, for example, in-frame linker sequence.

As used herein, the term “fusion protein” means a protein comprising a first polypeptide linearly connected, via peptide bonds, to a second, polypeptide.

As used herein, the term “CD95 fusion protein” refers to a polypeptide that is a functional equivalent of CD95 fused to heterologous polypeptide. The CD95 fusion protein will generally share at least one biological property in common with the CD95 polypeptide (as described above). An example of a CD95 fusion protein is a CD95 immunoadhesin.

As used herein, the term “immunoadhesin” designates antibody-like molecules which combine the binding specificity of a heterologous protein (an “adhesin”) with the effector functions of immunoglobulin constant domains. Structurally, the immunoadhesins comprise a fusion of an amino acid sequence with the desired binding specificity which is other than the antigen recognition and binding site of an antibody (i.e., is “heterologous”), and an immunoglobulin constant domain sequence. The adhesin part of an immunoadhesin molecule typically is a contiguous amino acid sequence comprising at least the binding site of a receptor or a ligand. The immunoglobulin constant domain sequence in the immunoadhesin may be obtained from any immunoglobulin, such as IgG-1, IgG-2, IgG-3, or IgG-4 subtypes, IgA (including IgA-1 and IgA-2), IgE, IgD or IgM. The immunoglobulin sequence preferably, but not necessarily, is an immunoglobulin constant domain (Fc region). Immunoadhesins can possess many of the valuable chemical and biological properties of human antibodies. Since immunoadhesins can be constructed from a human protein sequence with a desired specificity linked to an appropriate human immunoglobulin hinge and constant domain (Fc) sequence, the binding specificity of interest can be achieved using entirely human components. Such immunoadhesins are minimally immunogenic to the patient, and are safe for chronic or repeated use. In some embodiments, the Fc region is a native sequence Fc region. In some embodiments, the Fc region is a variant Fc region. In some embodiments, the Fc region is a functional Fc region. The CD95 portion and the immunoglobulin sequence portion of the CD95 immunoadhesin may be linked by a minimal linker. The immunoglobulin sequence preferably, but not necessarily, is an immunoglobulin constant domain. The immunoglobulin moiety in the chimeras of the present invention may be obtained from IgG1, IgG2, IgG3 or IgG4 subtypes, IgA, IgE, IgD or IgM, but preferably IgG1 or IgG3. As used herein, the term “Fc region” is used to define a C-terminal region of an immunoglobulin heavy chain, including native sequence Fc regions and variant Fc regions. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof.

Another example of a CD95 fusion protein is a fusion of the CD95 polypeptide with human serum albumin-binding domain antibodies (AlbudAbs) according to the AlbudAb™ Technology Platform as described in Konterman et al. 2012 AlbudAb™ Technology Platform-Versatile Albumin Binding Domains for the Development of Therapeutics with Tunable Half-Lives

Typically a CD95 fusion according to the invention may be APG101 which is developed by Apogenix™. APG101 is a fully human fusion protein consisting of the extracellular domain of the CD95 receptor and the Fc domain of an IgG antibody.

In some embodiments, the agent is an aptamer. Aptamers are a class of molecule that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by EXponential enrichment (SELEX) of a random sequence library. The random sequence library is obtainable by combinatorial chemical synthesis of DNA. In this library, each member is a linear oligomer, eventually chemically modified, of a unique sequence. Peptide aptamers consists of a conformationally constrained antibody variable region displayed by a platform protein, such as E. coli Thioredoxin A that are selected from combinatorial libraries by two hybrid methods.

In some embodiments the CD95 antagonist of the present invention is an inhibitor of CD95 expression (or CD95L expression).

An “inhibitor of expression” refers to a natural or synthetic compound that has a biological effect to inhibit the expression of a gene. Therefore, an “inhibitor of CD95 expression” denotes a natural or synthetic compound that has a biological effect to inhibit the expression of CD95 gene.

Inhibitors of gene expression for use in the present invention may be based on antisense oligonucleotide constructs. Anti-sense oligonucleotides, including anti-sense RNA molecules and anti-sense DNA molecules, would act to directly block the translation of CD95 mRNA by binding thereto and thus preventing protein translation or increasing mRNA degradation, thus decreasing the level of CD95, and thus activity, in a cell. For example, antisense oligonucleotides of at least about 15 bases and complementary to unique regions of the mRNA transcript sequence encoding CD95 can be synthesized, e.g., by conventional phosphodiester techniques and administered by e.g., intravenous injection or infusion. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732).

Small inhibitory RNAs (siRNAs) can also function as inhibitors of gene expression for use in the present invention. Gene expression can be reduced by contacting the tumor, subject or cell with a small double stranded RNA (dsRNA), or a vector or construct causing the production of a small double stranded RNA, such that gene expression is specifically inhibited (i.e. RNA interference or RNAi). Methods for selecting an appropriate dsRNA or dsRNA-encoding vector are well known in the art for genes whose sequence is known (e.g. see Tuschi, T. et al. (1999); Elbashir, S. M. et al. (2001); Hannon, G J. (2002); McManus, M T. et al. (2002); Brummelkamp, T R. et al. (2002); U.S. Pat. Nos. 6,573,099 and 6,506,559; and International Patent Publication Nos. WO 01/36646, WO 99/32619, and WO 01/68836).

Ribozymes can also function as inhibitors of gene expression for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of CD95 mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays.

Both antisense oligonucleotides and ribozymes useful as inhibitors of gene expression can be prepared by known methods. These include techniques for chemical synthesis such as, e.g., by solid phase phosphoramadite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

Antisense oligonucleotides siRNAs and ribozymes of the invention may be delivered in vivo alone or in association with a vector. In its broadest sense, a “vector” is any vehicle capable of facilitating the transfer of the antisense oligonucleotide siRNA or ribozyme nucleic acid to the cells. Preferably, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide siRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rouse sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art. Preferred viral vectors are based on non-cytopathic eukaryotic viruses in which non-essential genes have been replaced with the gene of interest. Non-cytopathic viruses include retroviruses (e.g., lentivirus), the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in KRIEGLER (A Laboratory Manual,” W.H. Freeman C.O., New York, 1990) and in MURRY (“Methods in Molecular Biology,” vol. 7, Humana Press, Inc., Cliffton, N.J., 1991). Preferred viruses for certain applications are the adeno-viruses and adeno-associated viruses, which are double-stranded DNA viruses that have already been approved for human use in gene therapy. The adeno-associated virus can be engineered to be replication deficient and is capable of infecting a wide range of cell types and species. It further has advantages such as, heat and lipid solvent stability; high transduction frequencies in cells of diverse lineages, including hematopoietic cells; and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression characteristic of retroviral infection. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion. Other vectors include plasmid vectors. Plasmid vectors have been extensively described in the art and are well known to those of skill in the art. See e.g., SANBROOK et al., “Molecular Cloning: A Laboratory Manual,” Second Edition, Cold Spring Harbor Laboratory Press, 1989. In the last few years, plasmid vectors have been used as DNA vaccines for delivering antigen-encoding genes to cells in vivo. They are particularly advantageous for this because they do not have the same safety concerns as with many of the viral vectors. These plasmids, however, having a promoter compatible with the host cell, can express a peptide from a gene operatively encoded within the plasmid. Some commonly used plasmids include pBR322, pUC18, pUC19, pRC/CMV, SV40, and pBlueScript. Other plasmids are well known to those of ordinary skill in the art. Additionally, plasmids may be custom designed using restriction enzymes and ligation reactions to remove and add specific fragments of DNA. Plasmids may be delivered by a variety of parenteral, mucosal and topical routes. For example, the DNA plasmid can be injected by intramuscular, intradermal, subcutaneous, or other routes. It may also be administered by intranasal sprays or drops, rectal suppository and orally. It may also be administered into the epidermis or a mucosal surface using a gene-gun. The plasmids may be given in an aqueous solution, dried onto gold particles or in association with another DNA delivery system including but not limited to liposomes, dendrimers, cochleate and microencapsulation.

By a “therapeutically effective amount” of CD95 antagonist as above described is meant a sufficient amount of the CD95 antagonist for the treatment of the Th17 mediated disease. It will be understood, however, that the total daily usage of the compounds and compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidential with the specific polypeptide employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

The CD95 antagonist of the invention may be combined with pharmaceutically acceptable excipients, and optionally sustained-release matrices, such as biodegradable polymers, to form pharmaceutical compositions. As used herein, the term “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms. Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The CD95 antagonist of the invention can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifusoluble agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active polypeptides in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media, which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject.

The invention will be further illustrated by the following figures and examples. However, these examples and figures should not be interpreted in any way as limiting the scope of the present invention.

### FIGURES

FIG. 1. High amounts of serum CD95L in SLE patients correspond to a homotrimeric ligand inducing endothelial transmigration of activated T lymphocytes. A. Soluble CD95L was dosed by ELISA in sera of newly diagnosed SLE patients and healthy donors. *** indicates P<0.0001 using a two-tailed student's t test. B. Sera from SLE patient were fractionated using size exclusion S-300-HR Sephacryl columns and CD95L was dosed by ELISA. Inset: CD95L was immunoprecipitated in fractions 40-46 and 76-78 and loaded in a 12% SDS-PAGE. Anti-CD95L immunoblot is depicted. C. Activated PBLs from healthy donors were incubated in presence of gel filtration fractions obtained in B and endothelial transmigration was assessed as described in Materials and Methods. Where indicated, fractions 76-78 were pre-incubated 30 minutes with the antagonist anti-CD95L mAb NOK-1 (10 μg/ml). D. CD95L, IL17 and CD4 expression levels were analyzed by Immunohistochemistry in inflamed skins of lupus patients or in healthy subjects (mammectomy). Numbers correspond to different patients. E. Densitometric analyses of CD95L and IL17 staining depicted in D revealed that the expression levels of these two markers vary in a correlated manner. F. The indicated human T-cell subsets were subject to transmigration assay in presence of sera taken from SLE patients or healthy donor as controls. Data were analyzed using Mann-Whitney U-test. ***P<0.001 G. Human T-cell subsets were subject to transmigration assay as above except in the lower chamber Fas-Fc was added at increasing concentrations in parallel with cl-CD95L. Data represent the mean of 4-5 individual donors±SD and were analyzed using a 2-way Anova. H. Transmigration of CD4 T-cell subsets was analyzed in Boyden chambers in presence or absence of cl-CD95L (200 ng/ml). I. Transmigration of human regulatory T-cells and Th17 cells was assessed by Boyden Chamber assay in presence or absence of cl-CD95L. Data was analyzed using a 2-way Anova. P values<0.05 was considered significant; *P<0.05, ***P<0.001

FIG. 2. In vivo administration of cl-CD95L preferentially attracts Th17 cells. Mice were injected once with cl-CD95L (200 ng) or vehicle, and 24 hrs later subject to examination. (A-B). Total cell counts for the peritoneal cavity (A) and spleen (B) were performed. (C-D). Differential white blood cell count was performed 24 hrs post injection. Peritoneal Exudate Cells (PEC) (C) and spleen (D) cells were subject to flow cytometry analysis to identify the percentage of infiltrated CD4⁺ cells. (E-I). PEC CD4⁺ cells were purified by AutoMACS separation and RNA prepared. Cells were subject to real-time PCR for (E) IL-17A, (F) IL-23R, (G) CCR6, (H) IFN-γ, and (I) FoxP3. Data presented are averages of groups of 6 mice±SD, with experiments repeated twice. Data were analyzed using the students t-Test, P values<0.05 was considered significant; *P<0.05, **P<0.01, ***P<0.001.

FIG. 3. CD95 implements a Death Domain-independent Ca′ response. A. CEM cells were stimulated with CD95L (100 ng/mL) and CD95 was immunoprecipitated. The immune complex was resolved by SDS/PAGE, and the indicated immunoblottings were performed. Total lysates were loaded as control. B. Parental Jurkat T cells, PLC-γl-deficient and its PLC-γl-reconstituted counterparts were loaded with the Ca²⁺ probe FuraPE3-AM (1 μM) and then stimulated with cl-CD95L (100 ng/mL, black arrow). Ratio images (F340/F380, R) were taken every 10 s and were normalized vs pre-stimulated values (R₀). Data represent mean±SD of R/R₀ measured in n cells. Inset: PLCγl-deficient Jurkat cells or its reconstituted counterpart was lysed and the expression levels of PLCγl and CD95 were evaluated by immunoblotting. Tubulin was used as a loading control. C. Cells were loaded with the Ca²⁺ probe FuraPE3-AM (1 μM) and then stimulated with cl-CD95L (100 ng/ml). Data were analyzed as described in B. Inset: Parental Jurkat cells (A3) or its counterparts lacking either FADD or caspase-8 were lysed and the expression levels of CD95, FADD and Caspase-8 were evaluated by immunoblotting.

### EXAMPLE

New insights into a Ca2+-inducing domain in CD95 sequence promoting Th17 cell transmigration and pathology progression in systemic lupus erythematosus.

Methods:

Patients and Ethics Statement

SLE patients fulfilled four or more of the 1982 revised ACR criteria for the disease. All clinical investigations were conducted according to the principles expressed in the Declaration of Helsinki. Blood was sampled from patients diagnosed with SLE after written consent was obtained from each individual. This study was approved by institutional review board at the Centre Hospitalier Universitaire de Bordeaux.

Antibodies Other Reagents

PHA, 3-[4,5-dimethylthiazol-2-yl]-2,5-diphenyltetrazolium bromide (MTT), protease and phosphatase inhibitors were purchased from Sigma-Aldrich (L'Isle-d'Abeau-Chesnes, France). Anti-CD95L mAb was from Cell Signaling Technology (Boston, Mass., USA). Recombinant IL-2 was obtained from PeproTech Inc. (Rocky Hill, N.J., USA). Anti-PLCγl was purchased from Millipore (St Quentin en Yvelines, France). Anti-CD95 mAbs (APO1-3) came from Enzo Life Sciences (Villeurbanne). PE-conjugated anti-human CD95 (DX2) mAb, anti-human FADD mAb (clone1), neutralizing anti-CD95L mAb (Nok1) were provided by BD Biosciences (Le Pont de Claix, France). Anti-caspase-8 (C15) and anti-Fas (C-20) mAbs were from Santa Cruz Biotechnology (Heidelberg, Germany). CD95-Fc, neutralizing anti-ICAM-1 and E-selectin mAbs.

Plasmids and Constructs

GFP-tagged human CD95 (hCD95) constructs were obtained by PCR and inserted in frame between the Nhe1 and EcoR1 sites of pEGFP-N1 (Clontech). Note that for all CD95 constructs the numbering takes into consideration the subtraction of 16 amino-acid of the signal peptide. Substitution of the cysteine at position 183 by a valine in hCD95⁽¹⁻²¹⁰⁾ was performed using the Quickchange Lightning Site-directed Mutagenesis kit (Agilent Technologies, Les Ulis, France) according to manufacturer instructions. Mouse full length CD95 (mCD95) was kindly provided by Dr Pascal Schneider (Universite de Lausanne, Lausanne, Switzerland). The mCD95 sequence lacking the signal peptide (SP-residues 1-21) was amplified by PCR. After digestion by BamHI/EcoRI, the amplicon was inserted into pcDNA3.1(+) vector in frame with SP sequence of the influenza virus hemagglutinin protein followed by a flag tag sequence and a 6 amino acid linker. The pTriEx-4 vector encoding for Myc-tagged full-length human PLCγl was a gift from Dr. Matilda Katan (Chester Beatty Laboratories, The Institute of Cancer Research, London, United Kingdom). Plasmids coding for full length CD95L and the secreted IgCD95L have been described elsewhere (Tauzin et al., 2011). All constructs were validated by sequencing on both strands (GATC Biotech, Constance, Germany).

Cell Lines and Peripheral Blood Lymphocytes

All cells were purchased from ATCC (Molsheim Cedex, France). T leukemic cell lines CEM, H9 and Jurkat were cultured in RPMI supplemented with 8% heat-inactivated FCS (v/v) and 2 mM L-glutamine at 37° C. in a 5% CO2 incubator. CEM-IRC cell expressing a low amount of plasma membrane CD95 was described in (Beneteau et al., 2007; Beneteau et al., 2008). HEK293 cells were cultured in DMEM supplemented with 8% heat-inactivated FCS and 2 mM L-glutamine at 37° C. in a 5% CO2 incubator. PBMCs (peripheral blood mononuclear cells) from healthy donors were isolated by Ficoll centrifugation, washed twice in PBS. Monocytes were removed by a 2 hours adherence step and the naive PBLs (peripheral blood lymphocytes) were incubated overnight in RPMI supplemented with 1 μg/ml of PHA. Cells were washed extensively and incubated in the culture medium supplemented with 100 units/ml of recombinant IL-2 for 5 days. Human umbilical vein endothelial cell (HUVEC) (Jaffe et al., 1973) were grown in human endothelial serum free medium 200 supplemented with LSGS (Low serum growth supplement) (Invitrogen, Cergy Pontoise, France). CEM-IRC cells were electroporated using BTM-830 electroporation generator (BTX Instrument Division, Harvard Apparatus) with 10 μg of DNA. 24 hours after electroporation, cells were treated for one week with 1 mg/mL of neomycin and then clones were isolated using limiting dilution.

Immunohistocytology

Skins from lupus patients were embedded in paraffin and cut into 4 μm sections. For CD4, CD8 and IL17 detection, Immunohistochemical staining was performed on the Discovery Automated IHC stainer using the Ventana OmniMap detection kit (Ventana Medical Systems, Tucson, Ariz., USA). The slides were rinsed with Ventana Tris-based Reaction buffer (Roche). Following deparaffination with Ventana EZ Prep solution (Roche) at 75° C. for 8 min, antigen retrieval was performed using Ventana proprietary, Tris-based buffer solution CC1 (pH8) antibody, at 95° C. to 100° C. for 48 min. Endogen peroxidase was blocked with Inhibitor-D 3% H2O2 (Ventana) for 10 min at 37° C. After rinsing, slides were incubated at 37° C. for 60 min with IL17 (Bioss), CD4 and CD8 (Dako), and secondary antibody: OmniMap HRP for 32 min (Roche). Signal enhancement was performed using the Ventana ChromoMap Kit Slides (biotin free detection system). For CD95L (BD Pharmigen) detection, antigen retrieval was performed using antigen unmasking solution pH 9 (Vector) at 95° C. for 40 min and endogenous peroxidase was blocked using 3% w/v hydrogen peroxide in methanol for 15 min. Slides were incubated in 5% BSA for 30 min at RT and then stained overnight at 4° C. Tissue sections were incubated with Envision+ system HRP-conjugated secondary antibodies for 30 min at RT and labeling was visualized by adding liquid DAB+. Sections were counterstained (hematoxylin) and mounted with DPX mounting medium. Using ImageJ software (IHC toolbox), densitometry analysis was undertaken on scanned slides to evaluate the amount of the different markers. The mean area for each marker was assessed and we determined if a correlation existed between the quantities of IL17-expressing cells and CD8⁺ T cells and the expression level of CD95L.

Mouse and Human CD4⁺ T-Cell Subset Generation

Animal experiments were subject to ethical review by the University of Nottingham were appropriate and conducted using PPL 40/3412 in accordance with the UK Home Office guidance and under ASPA (1986). For the generation of murine T-cell subsets, spleens were removed from C57Bl/6 mice and single cell suspensions prepared. CD4⁺ CD62L⁺ naïve cells were isolated using Miltenyi Biotec microbeads. Naïve human CD4⁺ T-cells were prepared using the Miltenyi Biotec naïve CD4⁺ T-cell isolation kit II, which are sorted produced a 99% pure population of CD4⁺ CD45RA⁺ cells. Purified cells were cultured in complete IMDM media all with α-CD3 (1 μg/ml), α-CD28 (2 μg/ml), and as follows; Th1 cells IL-12 (10 ng/ml) with α-IL-4 (10 μg/ml), Th2 cells IL-4 (10 ng/ml) and α-IFN-γ (10 μg/ml), Th17 cells Il-6 (10 ng/ml), TGF-β1 (2 ng/ml), α-IFN-γ (10 μg/ml) with α-IL-4 (10 μg/ml), and Tregs IL-2 (long/ml) TGF-β1 (5 ng/ml), α-IFN-γ (10 μg/ml) and α-IL-4 (10 μg/ml). Cells were converted to T-cell subsets over five days as outlined above. All cytokines were supplied by PeproTech (London, UK). Mouse CD3 (clone 2C11); human CD3 (UCHT1); mouse CD28 (37.51); human CD28 (CD28.2); mouse IL-4 (11B11); human IL-4 (MP4-25D2); mouse IFN-γ (XMG1.2); human IFN-γ B27 came from BD Pharmigen.

In Vivo Administration of Cl-CD95L

Female C57BL/6 mice (Harlan UK) aged between 8-10 weeks were placed in groups of 6 and administered IP. Twenty-four hours following injection, mice were sacrificed and periteonial cavities were washed with 5 ml of PBS/2% FCS, blood smears were prepared, and spleens were collected. Blood smears and cytospins of periteonal cells (PECs) were stained with Giemsa and differential counts performed. Single cell suspensions of spleens and PECs were prepared, cell counts performed, CD4+CD62− T-cells were isolated with Miltenyi microbeads and number of cells determined by trypan blue exclusion. All mouse experiments were performed under ethical approval from the University of Nottingham local animal ethics committee and adhering to UK Home Office guidelines under the Project License 40/3412.

Metalloprotease-Cleaved and Ig-Fused CD95L Production

Ig-CD95L was generated in the laboratory as described in (Tauzin et al., 2011). HEK 293 cells maintained in an 8% FCS-containing medium were transfected using Calcium/Phosphate precipitation method with 3 μg of empty plasmid or wild type CD95L-containing vector. 16 hours after transfection, medium was replaced by OPTI-MEM (Invitrogen) supplemented with 2 mM L-glutamine and 5 days later, media containing cleaved CD95L and exosome-bound full length CD95L were harvested. Dead cells and debris were eliminated through two steps of centrifugation (4500 rpm/15 minutes) and then exosomes were eliminated by an ultracentrifugation step (100000 g/2 hours).

Size Exclusion Chromatography

Sera from 4 different SLE patients (5·10⁷ cells) were filtrated using a 0.2 μm filter and then 5 ml was resolved using a mid range fractionation S300-HR Sephacryl column (GE Healthcare) equilibrated with PBS (pH 7.4). Using an AKTAprime purifier apparatus (GE Healthcare), fractions were harvested with a flow rate of 0.5 mL/min. Fifty fractions were harvested and analyzed by ELISA to quantify CD95L.

CD95L ELISA

Anti-CD95L ELISA (Diaclone, Besancon, France) was performed to accurately quantify the cleaved-CD95L present in sera following the manufacturer's recommendations.

Immunoprecipitation

T-cells (5×10⁷ cells per condition) were stimulated with Ig-CD95L or cl-CD95L (100 ng/mL) for indicated times at 37° C. Cells were lysed, incubated with AP01.3 (1 ug/mL) for 15 min at 4° C. and CD95 was immunoprecipitated using A/G protein-coupled magnetic beads (Ademtech, Pessac, France) for 1 h. After extensive washing, the immune complex was resolved by SDS-PAGE and immunoblotting was performed with indicated antibodies.

Immunoblot Analysis

Cells were lyzed for 30 minutes at 4° C. in lysis buffer (25 mM HEPES pH 7.4, 1% v/v Triton X-100, 150 mM NaCl, 2 mM EGTA supplemented with a mix of protease inhibitors (Sigma-Aldrich)). Protein concentration was determined by the bicinchoninic acid method (PIERCE, Rockford, Ill., USA) according to the manufacturer's protocol. Proteins were separated on a 12% SDS-PAGE and transferred to a nitrocellulose membrane (GE Healthcare, Buckinghamshire, England). The membrane was blocked 15 minutes with TBST (50 mM Tris, 160 mM NaCl, 0.05% v/v Tween 20, pH 7.8) containing 5% w/v dried skimmed milk (TBS™). Primary antibody was incubated overnight at 4° C. in TBS™. The membrane was intensively washed (TBST) and then the peroxydase-labeled anti-rabbit or anti-mouse (SouthernBiotech, Birmingham, Ala., US) was added for 45 minutes. The proteins were visualized with the enhanced chemiluminescence substrate kit (ECL, GE Healthcare).

Transendothelial Migration of Activated T Lymphocytes

After hydration of the Boyden chamber membranes containing 3 m pore size membranes (Millipore, Molsheim, France), activated T-lymphocytes (10⁶) were added to the top chamber on a confluent monolayer of HUVEC in a low serum (1%)-containing medium. The bottom chamber was filled with low serum (1%)-containing medium in presence or absence of 100 ng/ml of cl-CD95L. In experiments using human sera, 500 μl of serum from either healthy donors or SLE patients was added in the lower reservoir. Cells were cultured for 24 h at 37° C. in a 5% CO2, humidified incubator. Transmigrated cells were counted in the lower reservoir by flow cytometry using a standard of 2.5×10⁴ fluorescent beads (Flow-count, Beckman Coulter).

Endothelial Cell Adhesion Assay

Blocking antibodies were used against E-selectin and ICAM-1 in the CHEMICON® endothelial cell adhesion assay (Millipore). Briefly, after activation of the endothelial cell layer with TNF-α, anti-mouse Ig controls, anti-E-selectin or anti-ICAM-1 were added at final concentrations of 10 μg/ml. Thereafter calcein-AM-stained T-cell subsets were incubated for 24 hours and unbound cells are washed. Cells attached to the endothelium were assessed using fluorescence plate reader.

Real-Time qPCR

Single cell suspensions of spleens and PECs were prepared as described above. RNA was extracted from CD4 T-cells using phenol/chloroform. cDNA was prepared using the Promega GO-Script Reverse Transcription Kit and used in Real-Time PCR. Briefly, cDNA samples were subject to Taqman assay performed on a Roche Lightcycler. Results are reported as expression levels were calculated using the Act method relative to HPRT.

Video Imaging of the Calcium Response in Living Cells

Experiments on Parent Cell Lines

T cells were loaded with Fura-PE3-AM (1 μM) at room temperature for 30 min in Hank's Balanced Salt Solution (HBSS). After washing, the cells were incubated for 15 min in the absence of Fura-PE3-AM to complete de-esterification of the dye. Cells were placed in the temperature controlled chamber (37° C.) of an inverted epifluorescence microscope (Olympus IX70) equipped with an x40 UApo/340-1.15 W water-immersion objective (Olympus), and fluorescence micrograph images were captured at 510 nm and 12-bit resolution by a fast-scan camera (CoolSNAP fx Monochrome, Photometrics). To minimize UV light exposure, a 4×4 binning function was used. Fura-PE3 was alternately excited at 340 and 380 nm, and the ratios of the resulting images (emission filter at 520 nm) were produced at constant intervals (10 seconds). The Fura-PE3 ratio (Fᵣₐₜᵢₒ 340/380) images were analyzed offline with Universal Imaging software, including Metafluor and Metamorph. Fᵣₐₜᵢₒ reflects the intracellular Ca²⁺ concentration changes. Each experiment was repeated 3 times, and the average of more than 20 single-cell traces was analyzed.

Experiments on GFP-Expressing Cell Lines

Fluo2-AM was used, instead of Fura-PE3-AM for experiments with GFP-expressing cells, because GFP fluorescence disturbs Ca²⁺ measurement with Fura-PE3. As for Fura-PE3-AM, T cells were loaded with Fluo2-AM (1 μM) for 30 min in Hank's Balanced Salt Solution (HBSS) and then incubated for 15 min in the Fluo2-AM free HBSS to complete de-esterification of the dye.Ca²⁺ changes were evaluated by exciting Fluo2-AM-loaded cells at 535±35 nm. The values of the emitted fluorescence (605±50 nm) for each cell (F) were normalized to the starting fluorescence (F0) and reported as F/F0 (relative Ca²⁺_{[CYT]}). Only GFP-positive cells were considered.

Results:

Serum CD95L in Lupus Patients Promotes Endothelial Transmigration of Activated Th17 Cells

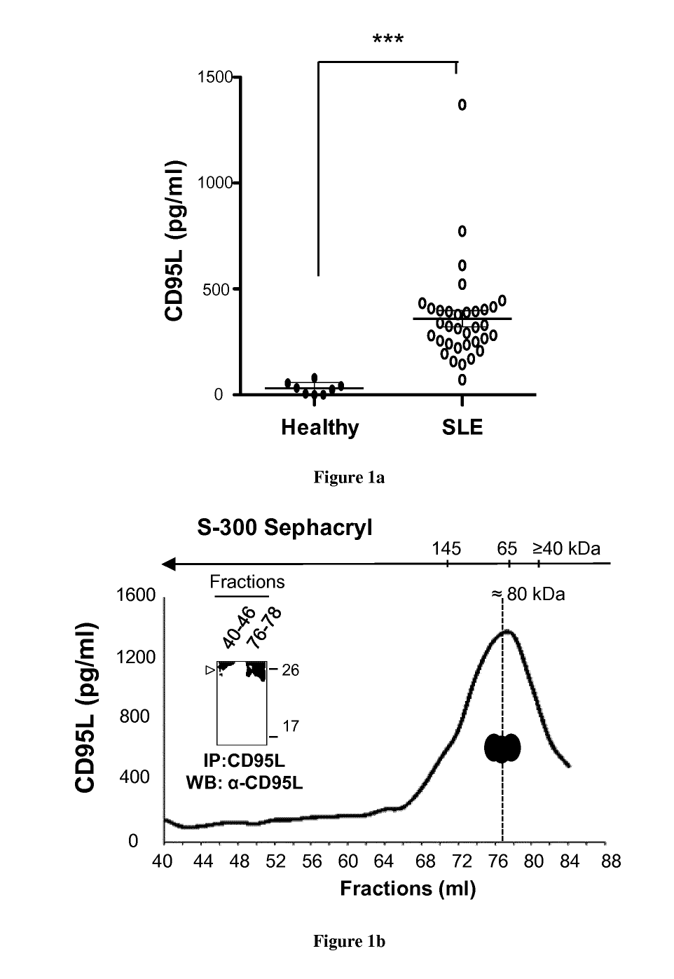

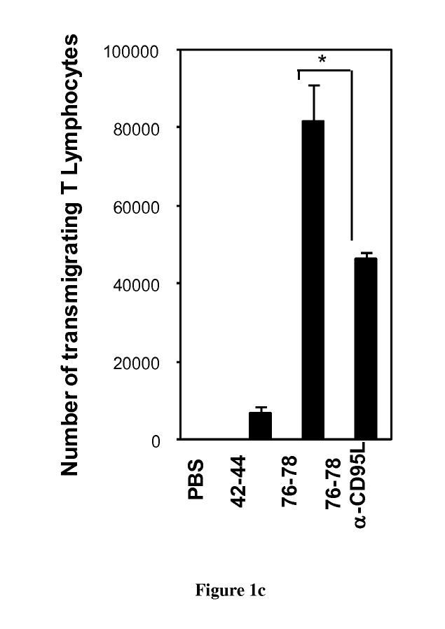

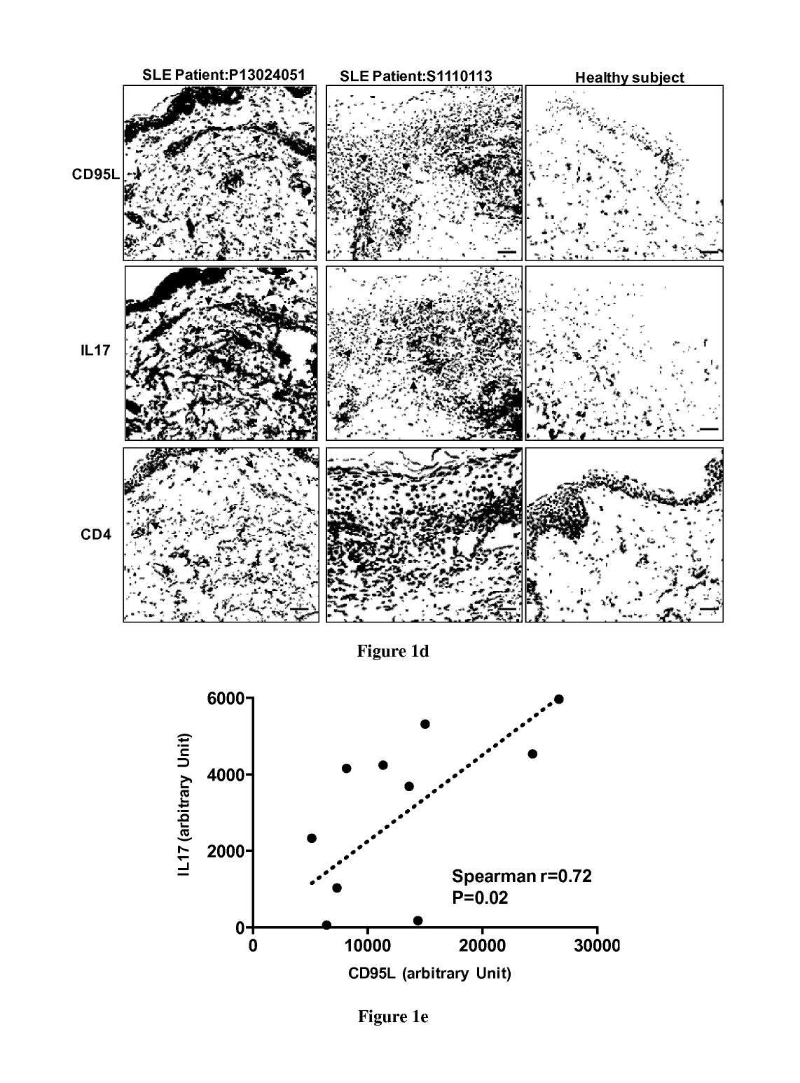

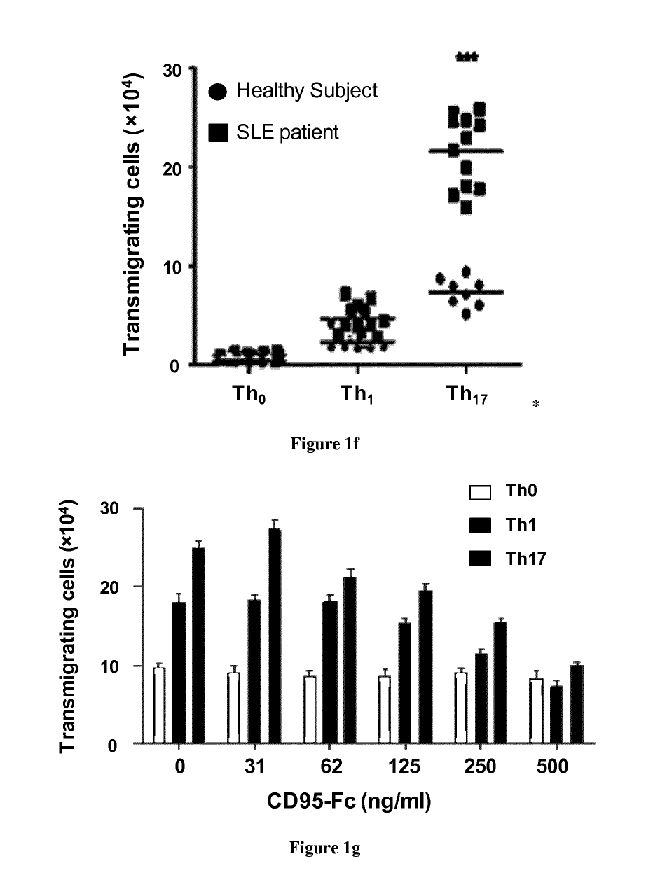

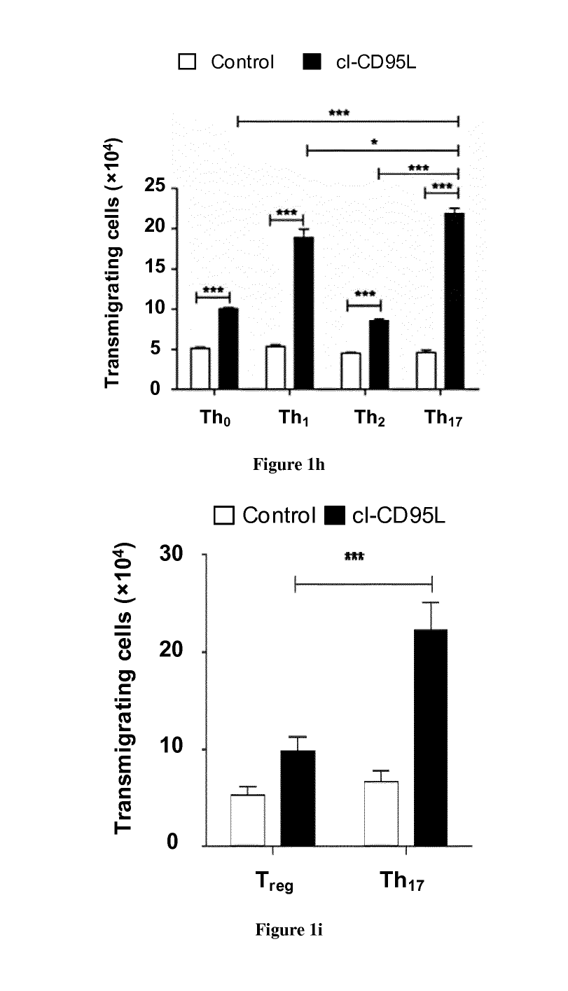

Recent reports suggest that a soluble form of CD95L increases in bronchoalveolar lavage fluid of patients suffering from acute respiratory distress syndrome (ARDS). Surprisingly, this soluble CD95L conserves its amino-terminal extracellular stalk region (amino acid residues 103 to 136), a region normally eliminated after shedding by metalloprotease of the membrane-bound CD95L (Herrero et al., 2011). Additionally under native conditions, this ARDS CD95L exhibited a hexameric stoichiometry and exerted a cytotoxic activity towards alveolar epithelial cells in lungs (Herrero et al., 2011). These results encouraged us to evaluate the stoichiometry of serum CD95L in SLE patients. First, we confirmed that soluble CD95L was significantly increased in the sera of 34 SLE patients as compared to 8 age-matched healthy donors (360±224.8 pg/ml in SLE patients vs 30.04±28.52 pg/ml in healthy subjects, P<0.0001) (FIG. 1A). Second, these sera were fractionated using size-exclusion chromatography and CD95L concentration was quantified in each eluted fraction (FIG. 1B). CD95L was detected in fractions 76 to 78, that contained proteins whose native molecular mass ranged between 75 and 80 kDa. This CD95L was next immunoprecipitated and resolved under denaturing/reducing conditions (SDS-PAGE) at 26 kDa (FIG. 1B) indicating that the serum CD95L accumulated in lupus patients corresponded to a homotrimeric ligand. Upon examination, we noted that functionally this serum CD95L retained the previously reported activity of cleaved-CD95L (cl-CD95L), as it promoted the transmigration of T lymphocytes across an endothelial monolayer (FIG. 1C). Specifically, significantly more activated T lymphocytes isolated from healthy donors exposed to fractions 76-78 crossed endothelial monolayers in comparison to lymphocytes exposed to fractions 42-44. These latter fractions, which contain exosome-bound CD95L (data not shown) failed to exert any pro-migratory effect (FIG. 1C). Furthermore, T-cell transmigration induced by fractions 76-78 was inhibited by up to 50% using a neutralizing anti-CD95L mAb (FIG. 1C) confirming that soluble CD95L in SLE patients plays a role in the endothelial transmigration of T lymphocytes. If T-cell infiltration is involved in tissue damage and Th17 cells contribute to this clinical outcome through a CD95-driven recruitment, we assumed that CD95L-expressing cells should be detected in the inflamed organs. Using immunohistochemistry, we evaluated the distribution of CD95L and IL17-expressing cells in lupus patients with skin lesions. Of note, CD95L and IL17 staining were observed in skin biopsies of lupus patients while they were undetectable in control skins (i.e., skins from breast reconstruction) (FIG. 1D). Moreover, CD95L was mainly detected on endothelium of blood vessels, which were surrounded by immune cell infiltration (FIG. 1D). Moreover, a densitometric analysis of lupus patients (n=10) highlighted that the amount of CD95L was correlated with the quantity of tissue-infiltrating IL17-expressing immune cells suggesting that this ligand may represent a chemoattractant for CD4⁺ Th17 cells (FIG. 1E). To further investigate if after cleavage by metalloprotease, CD95L exerted a chemoattractant activity toward all T-lymphocytes or selectively promoted migration of a sub-population, endothelial transmigration of naïve CD4⁺ T-cells isolated from healthy donors and subjected to in vitro differentiation was evaluated in presence or absence of healthy or SLE sera. As compared to healthy sera, sera from SLE patients triggered a moderate increase in Th1 transmigration while they dramatically enhanced endothelial transmigration of Th17 cells (FIG. 1F). More importantly, this transmigration process relied on CD95 signaling because pre-incubation of SLE sera with a decoy receptor (CD95-Fc) prevented Th17 cell migration in a dose-dependent manner (FIG. 1G).

Both Th1 and Th17 T-cells have been reported to accumulate in enflamed organs of lupus patients and lupus-prone mice contributing to disease pathogenesis. To eliminate a putative role played by other serum components in the observed phenomenon, we hereafter used a recombinant and homotrimeric version of CD95L. To this end, HEK 293 cells were transfected with a full-length CD95L-encoding vector and we used the metalloprotease-cleaved CD95L (cl-CD95L) contained in this supernatant (Tauzin et al., 2011). Similarly to serum CD95L in lupus patients, cl-CD95L was more efficient to promote the transmigration of Th1 and Th17 lymphocytes as compared to undifferentiated Th0 and differentiated Th2 cells (FIG. 1H). As imbalance of the Th17/T-regulatory (Treg) cell ratio in enflamed organs has been suggested to participate in autoimmune disorders and specifically lupus pathogenesis (Yang et al., 2009), we next evaluated the effect of cl-CD95L on the transmigration of Treg cells. As shown in FIG. 1I, cl-CD95L enhanced endothelial transmigration of Th17 T cells but failed to induce significant Treg transmigration indicating that the accumulation of Th17 cells at the expense of Treg cells in the inflamed tissues of lupus patients. These findings revealed that the higher levels of serum CD95L in SLE patients as compared to healthy donors could contribute to the accumulation of Th17 cells in inflamed organs.

Cellular recruitment and trafficking can be controlled by expression levels of adhesion molecules on lymphocytes and their molecular partners on endothelial cell surfaces. The expression of these molecules during an inflammatory response is a dynamic process, which increases or decreases the extravasation of immune cells into tissues. Recently, Th17 cells have been shown to accumulate in organs as a result of their interaction with E-selectin during rolling and ICAM-1-dependent arrest on activated endothelium (Alcaide et al., 2012). To address if these molecules contributed to the CD95-mediated endothelial T-cell migration of Th17 cells, we evaluated the expression level of key adhesion molecules on endothelial cells and differentiated Th cells in presence or absence of cl-CD95L. Of note, while an important amount of E-selectin was observed at the surface of HUVECs, no P-selectin was detected in these cells. Moreover, cl-CD95L did not alter the expression level of different adhesion molecules on HUVEC. By contrast, in presence of cl-CD95L, Th17 cells underwent up-regulation of P-selectin glycoprotein (PSGL-1), a ligand of E- and P-selectin, and ICAM-1 binding partner LFA-1. The expression level of these ligands remained unaffected in Th1 cells and tended towards a down-regulated state in Treg cells. Functionally the impact of PSGL-1 up-regulation in cl-CD95L-stimulated Th17 cells was evaluated by use of an E-selectin neutralizing mAb. Anti-E-selectin inhibited more efficiently Th17 cell transmigration when compared to similarly treated Th1 cells. Conversely blockade of ICAM-1/LFA-1 interactions by anti-ICAM-1 mAb impaired to a lesser extent both Th1 and Th17 cell migration across endothelial cells. These findings suggested that cl-CD95L promoted CD95-mediated Th17 cell transmigration by enhancing PSGL-1/E-selectin interaction.

Cl-CD95L Causes In Vivo a Rapid Accumulation of Th17 Cells.

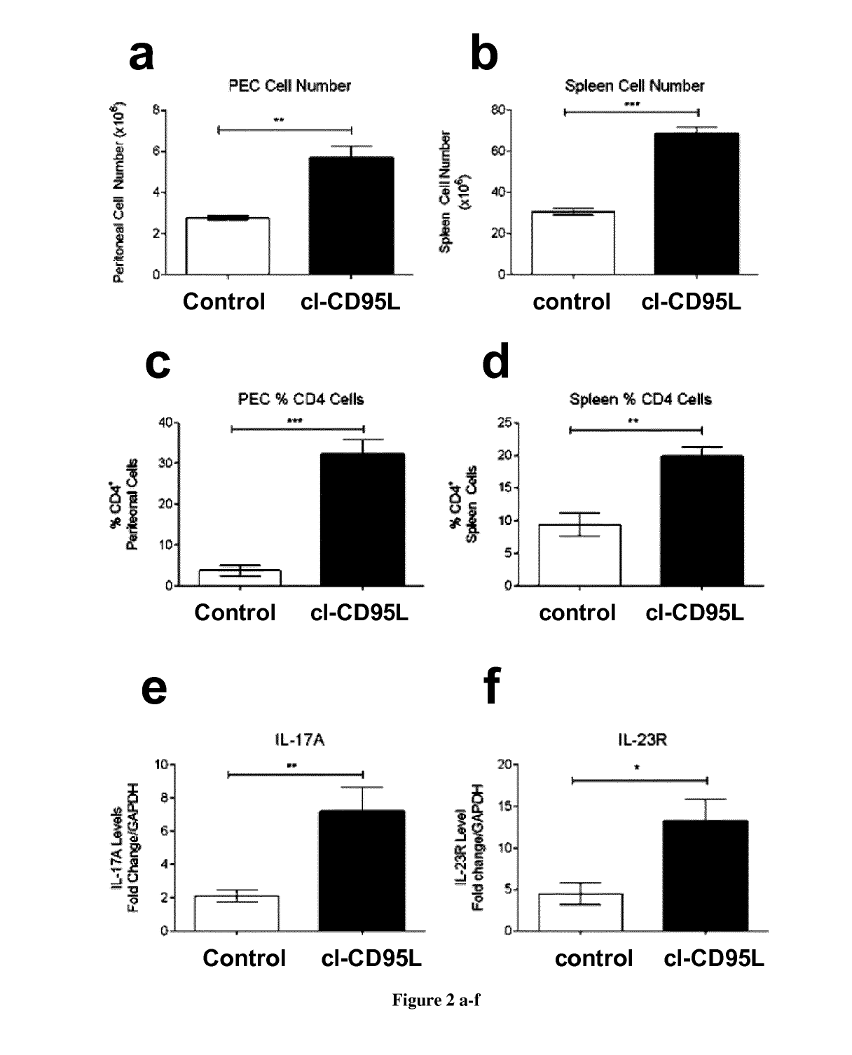

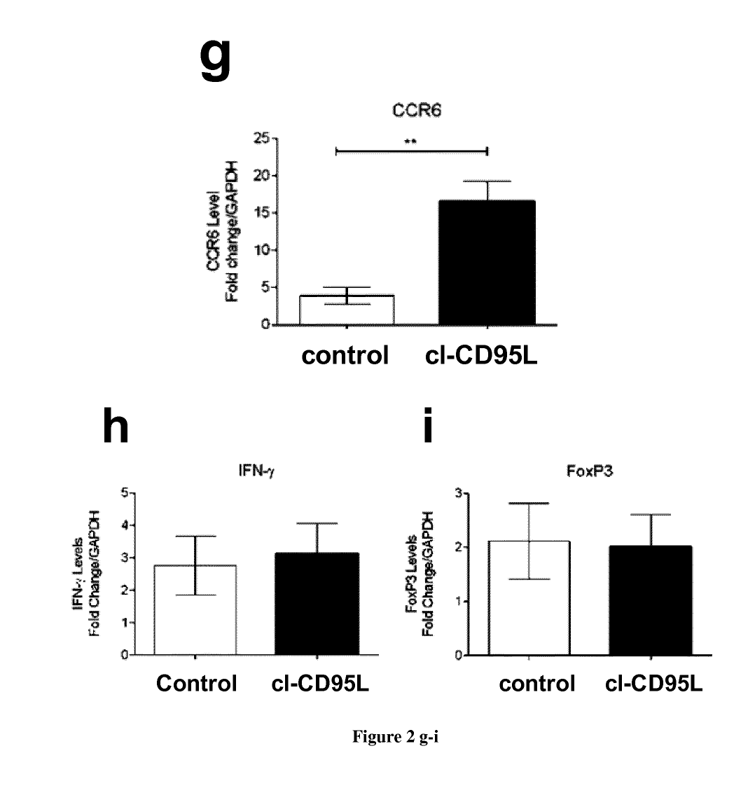

To confirm in vivo the chemoattractant ability of cl-CD95L towards Th17 cells, mice were injected intraperitoneally with a single dose of cl-CD95L or vehicle and 24 hours later, composition of T-cells infiltrating the peritoneal cavity (peritoneal exudate cells—PECs) and the spleen was examined. Total cell counts from the PEC and spleen revealed a significant increase in the number of lymphocytes in these compartments as compared to vehicle-injected mice (FIG. 2A-B). Loss of CD62L expression is associated with T-cell receptor engagement. Using this marker, we evaluated the amount of activated CD4⁺ T-cells (CD4⁺ CD62L⁻) recruited into the spleen and the peritoneal cavity of mice injected with or without cl-CD95L. We observed an increased amount of T cells recruited in the peritoneal cavity and the spleen upon injection of cl-CD95L as compared to control medium (FIG. 2C-D). Moreover, Q-PCR analyses of key markers of the Th17 lineage including IL-17 (FIG. 2E), IL-23R (FIG. 2F), and CCR6 (FIG. 2G), performed on these activated CD4+ T cells showed that cl-CD95L induced the recruitment of Th17 cells in these tissues. Furthermore, there was no increase in levels of IFN-γ (Th1 cells) and FoxP3 (Treg) levels upon examination (FIG. 2H-I) strongly supporting that cl-CD95L acted primarily as a potent chemotactic ligand to Th17 T cells.

CD95 Triggers a Death Domain-Independent Ca²⁺ Response

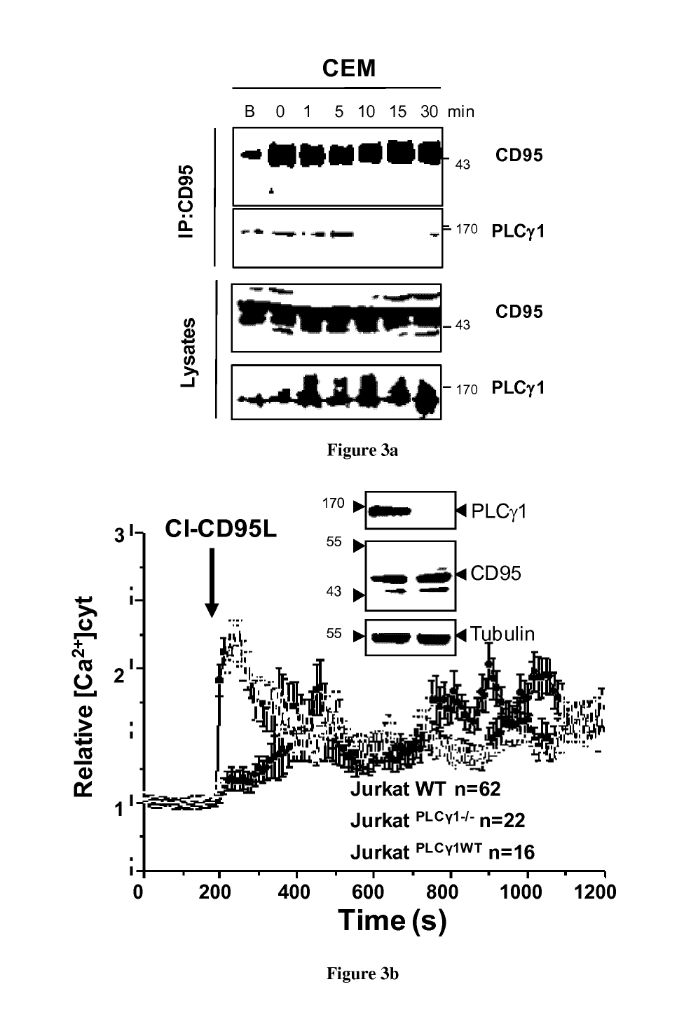

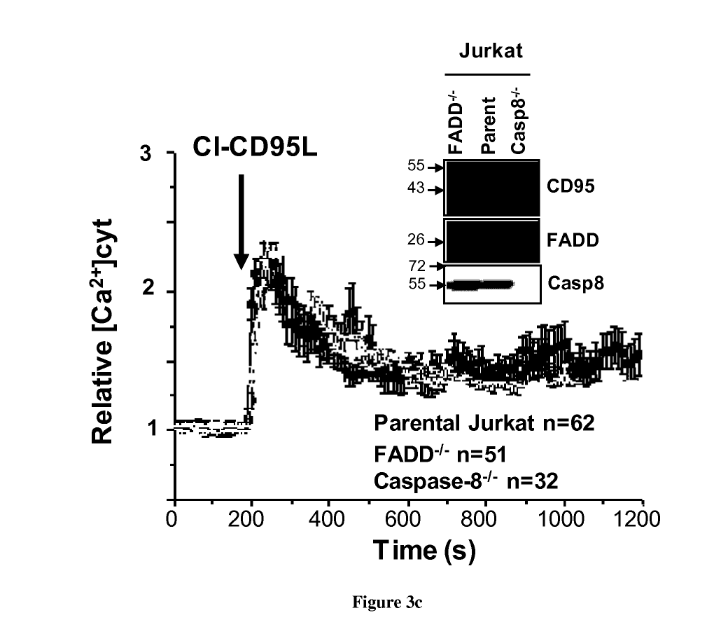

We recently showed that CD95 engagement evoked a Ca²⁺ response in activated T lymphocytes that transiently inhibited the apoptotic signal (Khadra et al., 2011) and promoted cell motility (Tauzin et al., 2011). These observations raised the question of whether inhibition of this CD95-mediated Ca²⁺ response can simultaneously inhibit cell migration and enhance or at least unalter the apoptotic signal. T-cells exposed to cl-CD95L rapidly formed a molecular complex containing the phospholipase Cγl (PLCγl) (FIG. 3A). Of note, the lack of this lipase in the T-cell line Jurkat caused a loss of the CD95-mediated Ca²⁺ signal, while reconstitution of these cells with wild type PLCγl restored a calcium response similar to that of the parental T-cell line (FIG. 3B). Next, we investigated if the main components of the DISC were instrumental in the CD95-mediated calcium signal. To this end, the calcium signal was assessed in FADD- and caspase-8-deficient Jurkat cells stimulated with cl-CD95L (FIG. 3C). Interestingly, although elimination of these molecules blocked the transmission of the apoptotic signaling pathway, it did not affect the CD95-mediated Ca²⁺ signal (FIG. 3C) indicating that PLCγl activation occurred independently of the DISC formation and the implementation of cell death signal.

Discussion:

Our study provides new insights into the cellular and molecular mechanisms by which metalloprotease-cleaved CD95L enhances inflammation in SLE patients. We show that transmembrane CD95L is ectopically expressed by endothelial cells covering blood vessels in the inflamed skins of lupus patients. More importantly, these CD95L⁺ vessels are surrounded by a massive immune infiltrate strongly suggesting that these structures may serve as “open doors” for pro-inflammatory cells among which Th17 cells. Exposed to cl-CD95L, these IL17-expressing cells up-regulate PSGL-1 and LFA-1, two adhesion molecules involved in rolling and tethering of leukocytes to endothelial cells. Of note, T cells with the highest levels of functional PSGL-1 also show the greatest capacity for effector cytokine secretion and for cytotoxic activity (Baaten et al., 2013). Therefore, cl-CD95L may fuel the inflammatory process not only by promoting the recruitment of activated Th1 and Th17 cells in inflamed tissues but also by altering the pattern of cytokine release in these organs. A recent Phase I/II clinical trial found that a decoy receptor (known as APG101) capable of blocking the CD95/CD95L interaction did not show any toxicity in humans suffering from glioblastoma (Tuettenberg et al., 2012). We may envision that this therapeutic agent may, in a short-term period, benefit lupus patients. We thus propose that selective inhibition of the CD95-mediated Ca²⁺ response will provide an excellent opportunity to block the pro-inflammatory activity of cl-CD95L in certain chronic inflammatory disorders.

### REFERENCES

Throughout this application, various references describe the state of the art to which this invention pertains. The disclosures of these references are hereby incorporated by reference into the present disclosure.
Alcaide, P., E. Maganto-Garcia, G. Newton, R. Travers, K. J. Croce, D. X. Bu, F. W. Luscinskas, and A. H. Lichtman. 2012. Difference in Th1 and Th17 lymphocyte adhesion to endothelium. J Immunol 188:1421-1430.
Baaten, B. J., A. M. Cooper, S. L. Swain, and L. M. Bradley. 2013. Location, Location, Location: The Impact of Migratory Heterogeneity on T Cell Function. Frontiers in immunology 4:311.
Beneteau, M., S. Daburon, J. F. Moreau, J. L. Taupin, and P. Legembre. 2007. Dominant-negative Fas mutation is reversed by down-expression of c-FLIP. Cancer Res 67:108-115.
Beneteau, M., M. Pizon, B. Chaigne-Delalande, S. Daburon, P. Moreau, F. De Giorgi, F. Ichas, A. Rebillard, M. T. Dimanche-Boitrel, J. L. Taupin, J. F. Moreau, and P. Legembre. 2008. Localization of Fas/CD95 into the lipid rafts on down-modulation of the phosphatidylinositol 3-kinase signaling pathway. Mol Cancer Res 6:604-613.
Chakrabandhu, K., Z. Herincs, S. Huault, B. Dost, L. Peng, F. Conchonaud, D. Marguet, H. T. He, and A. O. Hueber. 2007. Palmitoylation is required for efficient Fas cell death signaling. Embo J 26:209-220.
Crispin, J. C., M. Oukka, G. Bayliss, R. A. Cohen, C. A. Van Beek, I. E. Stillman, V. C. Kyttaris, Y. T. Juang, and G. C. Tsokos. 2008. Expanded double negative T cells in patients with systemic lupus erythematosus produce IL-17 and infiltrate the kidneys. J Immunol 181:8761-8766.
Cumberworth, A., G. Lamour, M. M. Babu, and J. Gsponer. 2013. Promiscuity as a functional trait: intrinsically disordered regions as central players of interactomes. Biochem J 454:361-369.
Desbarats, J., R. B. Birge, M. Mimouni-Rongy, D. E. Weinstein, J. S. Palerme, and M. K. Newell. 2003. Fas engagement induces neurite growth through ERK activation and p35 upregulation. Nat Cell Biol 5:118-125.
Feig, C., V. Tchikov, S. Schutze, and M. E. Peter. 2007. Palmitoylation of CD95 facilitates formation of SDS-stable receptor aggregates that initiate apoptosis signaling. Embo J 26:221-231.
Fouque, A., L. Debure, and P. Legembre. 2014. The CD95/CD95L signaling pathway: A role in carcinogenesis. Biochim Biophys Acta
Herrero, R., O. Kajikawa, G. Matute-Bello, Y. Wang, N. Hagimoto, S. Mongovin, V. Wong, D. R. Park, N. Brot, J. W. Heinecke, H. Rosen, R. B. Goodman, X. Fu, and T. R. Martin. 2011. The biological activity of FasL in human and mouse lungs is determined by the structure of its stalk region. The Journal of clinical investigation 121:1174-1190.
Ivanov, V. N., P. Lopez Bergami, G. Maulit, T. A. Sato, D. Sassoon, and Z. Ronai. 2003. FAP-1 association with Fas (Apo-1) inhibits Fas expression on the cell surface. Mol Cell Biol 23:3623-3635.
Jaffe, E. A., R. L. Nachman, C. G. Becker, and C. R. Minick. 1973. Culture of human endothelial cells derived from umbilical veins. Identification by morphologic and immunologic criteria. J Clin Invest 52:2745-2756.
Khadra, N., L. Bresson-Bepoldin, A. Penna, B. Chaigne-Delalande, B. Segui, T. Levade, A. M. Vacher, J. Reiffers, T. Ducret, J. F. Moreau, M. D. Cahalan, P. Vacher, and P. Legembre. 2011. CD95 triggers Orail-mediated localized Ca2+ entry, regulates recruitment of protein kinase C (PKC) beta2, and prevents death-inducing signaling complex formation. Proc Natl Acad Sci USA 108:19072-19077.
Kiaei, M., K. Kipiani, N. Y. Calingasan, E. Wille, J. Chen, B. Heissig, S. Rafii, S. Lorenzl, and M. F. Beal. 2007. Matrix metalloproteinase-9 regulates TNF-alpha and FasL expression in neuronal, glial cells and its absence extends life in a transgenic mouse model of amyotrophic lateral sclerosis. Exp Neurol 205:74-81.
Kirkin, V., N. Cahuzac, F. Guardiola-Serrano, S. Huault, K. Luckerath, E. Friedmann, N. Novac, W. S. Wels, B. Martoglio, A. O. Hueber, and M. Zornig. 2007. The Fas ligand intracellular domain is released by ADAM10 and SPPL2a cleavage in T-cells. Cell Death Differ 14:1678-1687.
Kischkel, F. C., S. Hellbardt, I. Behrmann, M. Germer, M. Pawlita, P. H. Krammer, and M. E. Peter. 1995. Cytotoxicity-dependent APO-1 (Fas/CD95)-associated proteins form a death-inducing signaling complex (DISC) with the receptor. Embo J 14:5579-5588.
Kleber, S., I. Sancho-Martinez, B. Wiestler, A. Beisel, C. Gieffers, O. Hill, M. Thiemann, W. Mueller, J. Sykora, A. Kuhn, N. Schreglmann, E. Letellier, C. Zuliani, S. Klussmann, M. Teodorczyk, H. J. Grone, T. M. Ganten, H. Sultmann, J. Tuttenberg, A. von Deimling, A. Regnier-Vigouroux, C. Herold-Mende, and A. Martin-Villalba. 2008. Yes and PI3K bind CD95 to signal invasion of glioblastoma. Cancer Cell 13:235-248.
Lai, Y. J., V. T. Lin, Y. Zheng, E. N. Benveniste, and F. T. Lin. 2010. The adaptor protein TRIPE antagonizes Fas-induced apoptosis but promotes its effect on cell migration. Mol Cell Biol 30:5582-5596.
Malleter, M., S. Tauzin, A. Bessede, R. Castellano, A. Goubard, F. Godey, J. Leveque, P. Jezequel, L. Campion, M. Campone, T. Ducret, G. Macgrogan, L. Debure, Y. Collette, P. Vacher, and P. Legembre. 2013. CD95L cell surface cleavage triggers a prometastatic signaling pathway in triple-negative breast cancer. Cancer Res 73:6711-6721.
Matsuno, H., K. Yudoh, Y. Watanabe, F. Nakazawa, H. Aono, and T. Kimura. 2001. Stromelysin-1 (MMP-3) in synovial fluid of patients with rheumatoid arthritis has potential to cleave membrane bound Fas ligand. J Rheumatol 28:22-28.
Motz, G. T., S. P. Santoro, L. P. Wang, T. Garrabrant, R. R. Lastra, I. S. Hagemann, P. Lal, M. D. Feldman, F. Benencia, and G. Coukos. 2014. Tumor endothelium FasL establishes a selective immune barrier promoting tolerance in tumors. Nat Med
O' Reilly, L. A., L. Tai, L. Lee, E. A. Kruse, S. Grabow, W. D. Fairlie, N. M. Haynes, D. M. Tarlinton, J. G. Zhang, G. T. Belz, M. J. Smyth, P. Bouillet, L. Robb, and A. Strasser. 2009. Membrane-bound Fas ligand only is essential for Fas-induced apoptosis. Nature 461:659-663.
O'Reilly, K. E., F. Rojo, Q. B. She, D. Solit, G. B. Mills, D. Smith, H. Lane, F. Hof nann, D. J. Hicklin, D. L. Ludwig, J. Baselga, and N. Rosen. 2006. mTOR inhibition induces upstream receptor tyrosine kinase signaling and activates Akt. Cancer Res 66:1500-1508.
Orlinick, J. R., K. B. Elkon, and M. V. Chao. 1997. Separate domains of the human fas ligand dictate self-association and receptor binding. J Biol Chem 272:32221-32229.
Sato, T., S. Irie, S. Kitada, and J. C. Reed. 1995. FAP-1: a protein tyrosine phosphatase that associates with Fas. Science 268:411-415.
Schulte, M., K. Reiss, M. Lettau, T. Maretzky, A. Ludwig, D. Hartmann, B. de Strooper, O. Janssen, and P. Saftig. 2007. ADAM10 regulates FasL cell surface expression and modulates FasL-induced cytotoxicity and activation-induced cell death. Cell Death Differ 14:1040-1049.
Shin, M. S., N. Lee, and I. Kang. 2011. Effector T-cell subsets in systemic lupus erythematosus: update focusing on Th17 cells. Curr Opin Rheumatol 23:444-448.
Siegel, R. M., J. K. Frederiksen, D. A. Zacharias, F. K. Chan, M. Johnson, D. Lynch, R. Y. Tsien, and M. J. Lenardo. 2000. Fas preassociation required for apoptosis signaling and dominant inhibition by pathogenic mutations. Science 288:2354-2357.
Steinmetz, O. M., J. E. Turner, H. J. Paust, M. Lindner, A. Peters, K. Heiss, J. Velden, H. Hopfer, S. Fehr, T. Krieger, C. Meyer-Schwesinger, T. N. Meyer, U. Helmchen, H. W. Mittrucker, R. A. Stahl, and U. Panzer. 2009. CXCR3 mediates renal Th1 and Th17 immune response in murine lupus nephritis. J Immunol 183:4693-4704.
Suda, T., T. Takahashi, P. Golstein, and S. Nagata. 1993. Molecular cloning and expression of the Fas ligand, a novel member of the tumor necrosis factor family. Cell 75:1169-1178.
Tauzin, S., B. Chaigne-Delalande, E. Selva, N. Khadra, S. Daburon, C. Contin-Bordes, P. Blanco, J. Le Seyec, T. Ducret, L. Counillon, J. F. Moreau, P. Hofman, P. Vacher, and P. Legembre. 2011. The naturally processed CD95L elicits a c-yes/calcium/PI3K-driven cell migration pathway. PLoS Biol 9:e1001090.
Tuettenberg, J., M. Seiz, K. M. Debatin, W. Hollburg, M. von Staden, M. Thiemann, B. Hareng, H. Fricke, and C. Kunz. 2012. Pharmacokinetics, pharmacodynamics, safety and tolerability of APG101, a CD95-Fc fusion protein, in healthy volunteers and two glioma patients. International immunopharmacology 13:93-100.
Vargo-Gogola, T., H. C. Crawford, B. Fingleton, and L. M. Matrisian. 2002. Identification of novel matrix metalloproteinase-7 (matrilysin) cleavage sites in murine and human Fas ligand. Arch Biochem Biophys 408:155-161.
Vives, E., P. Brodin, and B. Lebleu. 1997. A truncated HIV-1 Tat protein basic domain rapidly translocates through the plasma membrane and accumulates in the cell nucleus. J Biol Chem 272:16010-16017.
Wang, Y., S. Ito, Y. Chino, D. Goto, I. Matsumoto, H. Murata, A. Tsutsumi, T. Hayashi, K. Uchida, J. Usui, K. Yamagata, and T. Sumida. 2010. Laser microdissection-based analysis of cytokine balance in the kidneys of patients with lupus nephritis. Clin Exp Immunol 159:1-10.
Yang, J., Y. Chu, X. Yang, D. Gao, L. Zhu, X. Yang, L. Wan, and M. Li. 2009. Th17 and natural Treg cell population dynamics in systemic lupus erythematosus. Arthritis Rheum 60:1472-1483.

1. A method of treating a Th17-mediated disease in a subject in need thereof comprising administering to the subject a therapeutically effective amount of a CD95 antagonist.

2. The method of claim 1 wherein the Th17-mediated disease is selected from the group consisting of autoimmune diseases, inflammatory diseases, osteoclasia, and transplantation rejection of cells, tissue and organs.

3. The method of claim 1 wherein the Th17-mediated disease are is selected from the group consisting of Behçet's disease, polymyositis/dermatomyositis, autoimmune cytopenias, autoimmune myocarditis, primary liver cirrhosis, Goodpasture's syndrome, autoimmune meningitis, Sjögren's syndrome, systemic lupus erythematosus, Addison's disease, alopecia greata, ankylosing spondylitis, autoimmune hepatitis, autoimmune mumps, Crohn's disease, insulin-dependent diabetes mellitus, dystrophic epidermolysis bullosa, epididymitis, glomerulonephritis, Graves' disease, Guillain-Barré syndrome, Hashimoto's disease, hemolytic anemia, multiple sclerosis, myasthenia gravis, pemphigus vulgaris, psoriasis, rheumatic fever, rheumatoid arthritis, sarcoidosis, scleroma, spondyloarthropathy, thyroiditis, vasculitis, vitiligo, myxedema, pernicious anemia and ulcerative colitis.

4. The method of claim 1 wherein the CD95 antagonist is an antibody having specificity for soluble CD95L or CD95.

5. The method of claim 4 wherein the antibody is capable of inhibiting binding of CD95 to soluble CD95L, which antibody binds to an epitope located within a region of CD95, which region of CD95 binds to soluble CD95L.

6. The method of claim 4 wherein the antibody is capable of binding to an epitope located within a region of CD95, which region of CD95 is involved the oligomerisation of the receptor.

7. The method of claim 5 wherein the antibody binds to the cysteine-rich domain 1 of CD95.

8. The method of claim 6 wherein the antibody binds to a region delimitated between the amino acid at position 43 and the amino acid at position 66 in CD95.

9. The method of claim 4 wherein the antibody is a chimeric, humanized or human antibody.

10. The method of claim 4 wherein the antibody is a single domain antibody.

11. The method of claim 1 wherein the CD95 antagonist is a polypeptide which comprises consecutive amino acids having a sequence which corresponds to the sequence of at least a portion of an extracellular domain of CD95, which portion binds to soluble CD95L.

12. The method of claim 11 wherein the polypeptide corresponds to an extracellular domain of CD95.

13. The method of claim 11 wherein the polypeptide is fused to a heterologous polypeptide.

14. The method of claim 11 wherein the polypeptide is fused to an immunoglobulin constant domain (Fc region).

15. The method of claim 11 wherein the polypeptide is an aptamer.

16. The method of claim 1 wherein the CD95 antagonist is an inhibitor of CD95 expression or CD95L expression.

17. The method of claim 16 wherein the inhibitor of CD95 expression is a siRNA.
